(19) United States
(12) Patent Application Publication
(10) Pub. No.: US 2025/0169690 A1
(43) Pub. Date: May 29, 2025

(54) ENDOSCOPE

(71) Applicant: OLYMPUS MEDICAL SYSTEMS CORP., Tokyo (JP)

(72) Inventors: Sho Saito, Hachioji-shi (JP); Yuichi Wataya, Hachioji-shi (JP)

(73) Assignee: OLYMPUS MEDICAL SYSTEMS CORP., Tokyo (JP)

(21) Appl. No.: 18/821,204

(22) Filed: Aug. 30, 2024

Related U.S. Application Data
(60) Provisional application No. 63/603,143, filed on Nov. 28, 2023.

(51) Int. Cl. A61B 1/05; A61B 1/00

(52) U.S. Cl. CPC A61B 1/05; A61B 1/00064; A61B 1/00114

(57) ABSTRACT

Provided is an endoscope capable of increasing an adhering strength between a signal cable and a holding frame.

An endoscope including: an imaging unit; and a holding frame having a tubular shape, holding the imaging unit, and extending in an optical axis direction, wherein the imaging unit includes an imaging element disposed in the holding frame, an imaging board connected to the imaging element, a signal cable extending in the optical axis direction, and a protective member having a tubular shape, surrounding the signal cable, and extending in the optical axis direction, wherein a distal end of the protective member faces the holding frame in the optical axis direction and is adhered to the holding frame, wherein a distal end portion of the protective member which is a portion on an object side including the distal end has a first distal end portion and a second distal end portion, wherein the first distal end portion is a part of the distal end portion in a circumferential direction, and the second distal end portion is another part of the distal end portion in the circumferential direction, and wherein a thickness of the first distal end portion in a radial direction is greater than a thickness of the second distal end portion in the radial direction.

Priority is claimed on U.S. Provisional Application No. 63/603,143, filed Nov. 28, 2023, the content of which is incorporated herein by reference.

### BACKGROUND OF THE INVENTION

### Field of the Invention

The present invention relates to an endoscope.

### Description of Related Art

An endoscope having a configuration in which a signal cable connected to an imaging element is adhesively fixed to an elastic cable attaching frame is known (for example, Patent Document 1). In the endoscope having such a configuration, even when bending stress is applied to the signal cable, the cable attaching frame is elastically deformed, thereby reducing the stress applied to the signal cable. Therefore, the connection between the imaging element and the signal cable can be stabilized.

### PATENT DOCUMENTS

[Patent Document 1] Japanese Unexamined Patent Application, First Publication No. 2012-170765

### SUMMARY OF THE INVENTION

In the above-mentioned endoscope, it is difficult to increase an adhering strength between the signal cable and the cable attaching frame in an optical axis direction, and thus, for example, when an imaging unit equipped with the imaging element and the signal cable is removed from an insertion section of the endoscope during maintenance of the endoscope, if the signal cable is pulled in the optical axis direction, there is a risk that the imaging unit will come off the cable attaching frame.

The present invention is made in view of the above circumstances, and an object of the present invention is to provide an endoscope capable of increasing an adhering strength between a signal cable and a holding frame.

In order to achieve the above-mentioned object, according to one aspect of the present invention, there is provided an endoscope including: an imaging unit; and a holding frame having a tubular shape, holding the imaging unit, and extending in an optical axis direction, wherein the imaging unit includes an imaging element disposed in the holding frame, an imaging board connected to the imaging element, a signal cable extending in the optical axis direction, and a protective member having a tubular shape, surrounding the signal cable, and extending in the optical axis direction, wherein a distal end of the protective member faces the holding frame in the optical axis direction and is adhered to the holding frame, wherein a distal end portion of the protective member which is a portion on an object side including the distal end has a first distal end portion and a second distal end portion, wherein the first distal end portion is a part of the distal end portion in a circumferential direction, and the second distal end portion is another part of the distal end portion in the circumferential direction, and wherein a thickness of the first distal end portion in a radial direction is greater than a thickness of the second distal end portion in the radial direction.

According to the present invention, it is possible to provide an endoscope capable of increasing an adhering strength between a signal cable and a holding frame.

### BRIEF DESCRIPTION OF THE DRAWINGS

FIG. 1 is a perspective view showing an endoscope system according to a first embodiment.

FIG. 2 is a perspective view showing an insertion section of the first embodiment.

FIG. 3 is a first cross-sectional view showing the insertion section of the first embodiment.

FIG. 4 is a second cross-sectional view showing the insertion section of the first embodiment.

FIG. 5 is a cross-sectional view showing a part of an imaging unit of the first embodiment.

FIG. 6 is a cross-sectional view showing a part of the imaging unit of the first embodiment, along line VI-VI in FIG. 5.

FIG. 7 is a cross-sectional view showing a part of an imaging unit of a first modification example of the first embodiment.

FIG. 8 is a cross-sectional view showing a part of an imaging unit of a second modification example of the first embodiment.

FIG. 9 is a cross-sectional view showing a part of an imaging unit of a third modification example of the first embodiment.

FIG. 10 is a cross-sectional view showing a part of an imaging unit of a fourth modification example of the first embodiment.

FIG. 11 is a cross-sectional view showing an imaging unit of a fifth modification example of the first embodiment.

FIG. 12 is a cross-sectional view showing a part of an imaging unit of a sixth modification example of the first embodiment.

FIG. 13 is a cross-sectional view showing a part of an imaging unit of a second embodiment.

FIG. 14 is a cross-sectional view showing a part of an imaging unit of a modification example of the second embodiment.

FIG. 15 is a cross-sectional view showing an insertion section of a third embodiment.

FIG. 16 is a cross-sectional view showing an insertion section of a modification example of the third embodiment.

FIG. 17 is a cross-sectional view showing an insertion section of a fourth embodiment.

FIG. 18 is a cross-sectional view showing an imaging unit of a modification example of the fourth embodiment.

FIG. 19 is a cross-sectional view showing an insertion section of a fifth embodiment.

FIG. 20 is a perspective view showing a unit holding frame of a sixth embodiment.

FIG. 21 is a cross-sectional view showing a part of an imaging unit of a sixth embodiment.

### DETAILED DESCRIPTION OF THE INVENTION

Hereinafter, endoscopes according to embodiments of the present invention will be described with reference to the drawings. The scope of the present invention is not limited to the following embodiments, and modifications can be made as desired within the scope of the technical concept of the present invention. In addition, in the drawings below, the scale and number of each structure may differ from those of the actual structure in order to make each configuration easier to understand.

In the drawings, a Z axis is shown as appropriate. The Z axis is a direction in which a central axis J extends in the embodiments which will be described below. The central axis J shown as appropriate in the drawings is a virtual axis. The central axis J is a central axis of an imaging unit. In the present embodiment, the central axis J overlaps an optical axis of an optical unit. The central axis J does not have to overlap the optical axis of the optical unit. In the following description, the direction in which the central axis J extends, that is, a direction parallel to the Z axis, is referred to as an “optical axis direction.” In the optical axis direction, a side (+Z side) toward which an arrow of the Z axis points is an “object side (distal side),” and a side (−Z side) opposite to the side toward which the arrow of the Z axis points is a “proximal end side (proximal side).”

In the following description, a radial direction centered on the central axis J is simply referred to as a “radial direction.” A circumferential direction centered on the central axis J is simply referred to as a “circumferential direction.” The circumferential direction is indicated by an arrow θ in the drawings. A side in the circumferential direction toward which the arrow θ points is referred to as “one side in the circumferential direction.” A side opposite to the side in the circumferential direction toward which the arrow θ points is referred to as the “other side in the circumferential direction.” The one side in the circumferential direction is a side (+θ side) that proceeds clockwise around the central axis J when viewed from the object side. The other side in the circumferential direction is a side (−θ side) that proceeds counterclockwise around the central axis J when viewed from the object side.

### First Embodiment

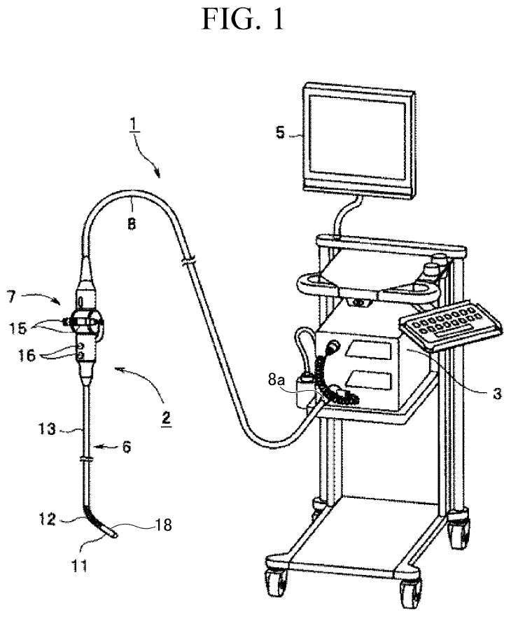

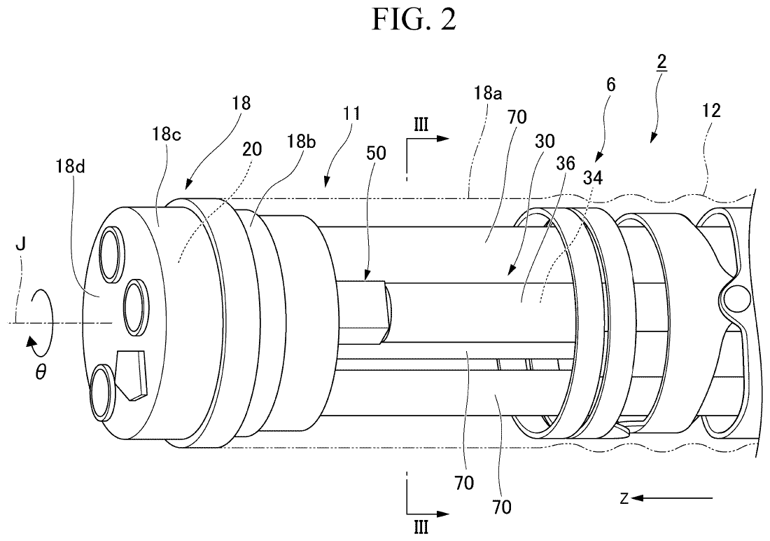

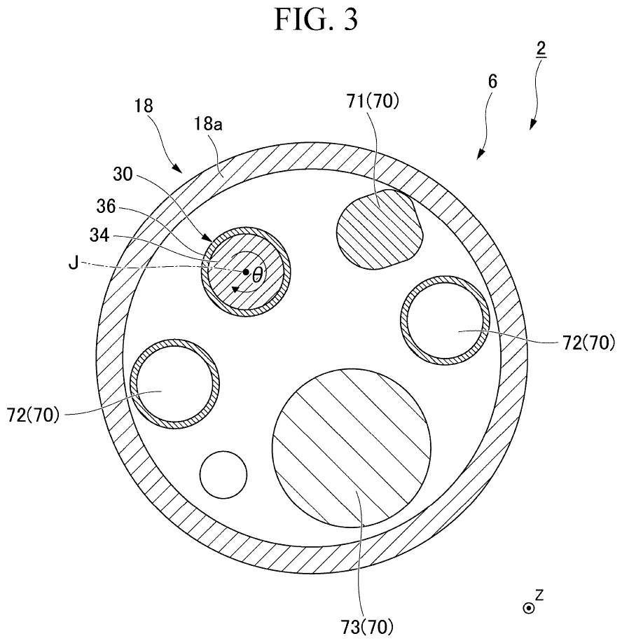

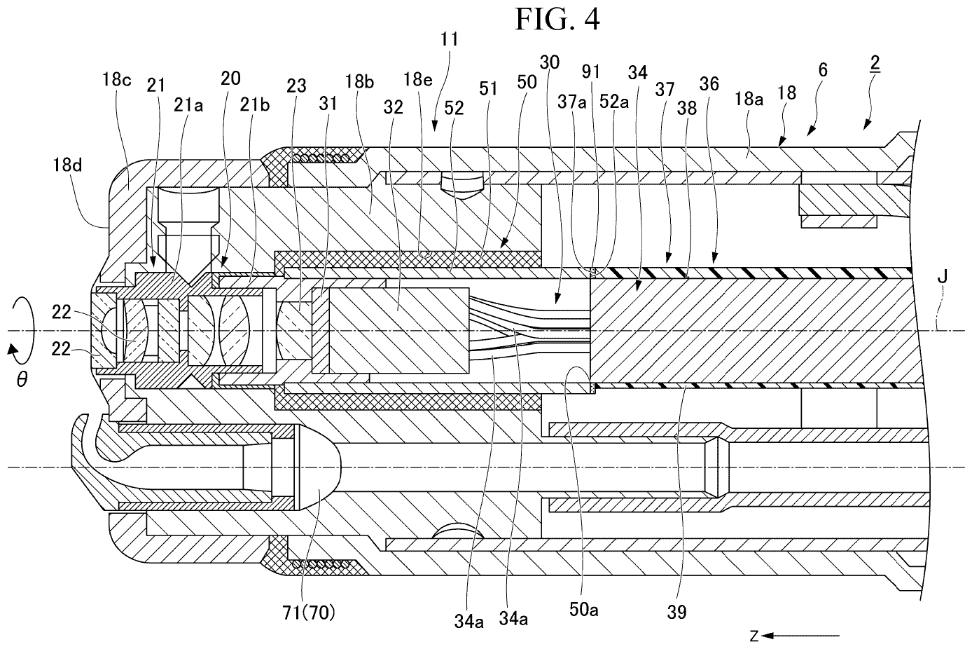

FIG. 1 is a perspective view showing an endoscope system 1 according to the present embodiment. FIG. 2 is a perspective view showing an insertion section 6 of the present embodiment. FIG. 3 is a first cross-sectional view showing the insertion section 6 of the present embodiment. FIG. 4 is a second cross-sectional view showing the insertion section of the present embodiment.

The endoscope system 1 shown in FIG. 1 is constituted by an endoscope 2 capable of capturing an image of a subject and generating the image of the subject, a processor 3 to which the endoscope 2 is detachably connected, and a monitor 5 that displays an image signal generated by the processor 3 as an image of the subject.

The endoscope 2 includes an elongated insertion section 6, an operation section 7 continuously provided on the proximal end side of the insertion section 6, and a universal cable 8 extending from the operation section 7 and connected to the processor 3. The use of the endoscope 2 of the present embodiment is not particularly limited and may be, for example, a medical endoscope or an industrial endoscope.

The insertion section 6 is a section that is inserted, for example, into the body of the subject when observing the subject. The insertion section 6 is inserted into the subject. The insertion section 6 is constituted by an insertion distal end portion 11, a bending portion 12, and a rigid tube portion 13 which are continuously provided in that order from the object side, which is a distal end in a direction of insertion into the subject, toward the proximal end side connected to the operation section 7. The insertion distal end portion 11 and the rigid tube portion 13 are formed of a metal tube made of stainless steel or the like. As shown in FIG. 2, in the present embodiment, the insertion distal end portion 11 has a substantially cylindrical shape and extends in the optical axis direction. The insertion distal end portion 11 includes a case 18, an optical unit 20, an imaging unit 30, a holding frame 50, and a plurality of accommodation objects 70. That is, the insertion section 6 has the case 18 and the imaging unit 30. The endoscope 2 includes the imaging unit 30 and the holding frame 50.

The case 18 accommodates therein the optical unit 20, the imaging unit 30, the holding frame 50, and the plurality of accommodation objects 70. That is, the imaging unit 30, the holding frame 50, and the plurality of accommodation objects 70 are accommodated in the insertion section 6. The case 18 holds the optical unit 20, the imaging unit 30, the holding frame 50, and the plurality of accommodation objects 70. In the present embodiment, the case 18 has a substantially cylindrical shape and extends in the optical axis direction. The case 18 has a tubular portion 18a, a holding portion 18b, and a distal end case 18c.

The tubular portion 18a has a substantially cylindrical shape and extends in the optical axis direction, and the tubular portion 18a is open on both sides in the optical axis direction. As shown in FIG. 3, a signal cable 34 and each of the plurality of accommodation objects 70 pass through the inside of the tubular portion 18a in the optical axis direction.

As shown in FIG. 4, the holding portion 18b has a substantially circular ring shape and extends in the optical axis direction. The holding portion 18b holds the optical unit 20, the imaging unit 30, the holding frame 50, and the plurality of accommodation objects 70. The holding portion 18b is disposed in the tubular portion 18a. The holding portion 18b is fixed to the inner circumferential surface of the tubular portion 18a. The holding portion 18b is provided with a recess 18e. The recess 18e is a recession recessed outward from the inner circumferential surface of the holding portion 18b in the radial direction. The recess 18e is open on the proximal end side (−Z side). When viewed in the optical axis direction, the recess 18e has a substantially tetragonal ring shape.

The distal end case 18c has a substantially cylindrical shape and protrudes in the optical axis direction. The distal end case 18c surrounds the optical unit 20 from the outside in the radial direction. The distal end case 18c is fixed to the end portion of the tubular portion 18a on the object side (+Z side). As shown in FIG. 2, the distal end case 18c has a distal end surface 18d. The distal end surface 18d is a portion of the outer surface of the insertion distal end portion 11 which faces the object side. A distal end opening portion, an observation window, an illumination window, and the like which are well known are formed in the distal end surface 18d.

The signal cable 34 (see FIG. 2 and the like) that electrically connects the imaging unit 30 to the operation section 7 and the processor 3, a light guide bundle (not shown) that transmits illumination light to the insertion distal end portion 11, and the like are inserted into the bending portion 12 and the rigid tube portion 13 shown in FIG. 1. The endoscope 2 of the present embodiment is a rigid endoscope in which the rigid tube portion 13 is disposed closer to the proximal end side than the bending portion 12, but is not limited to this, and the endoscope 2 may be a flexible endoscope in which a flexible tube portion with flexibility is disposed closer to the proximal end side than the bending portion 12.

The operation section 7 is provided with an angle lever 15 for remotely manipulating the bending portion 12 and various switches 16 for adjusting a focal position of an optical image of the subject which is formed by a light source device (not shown) provided in the processor 3 and the optical unit 20. The angle lever 15 is a manipulation means for manipulating the bending portion 12 such that the bending portion 12 bends in four directions, that is, upward, downward, leftward, and rightward. The bending portion 12 is not limited to being bendable in the four directions, that is, upward, downward, leftward, and rightward, but may be bendable in two directions, that is, only upward and downward, or only leftward and rightward, for example.

The signal cable 34 (see FIG. 2 and the like), the light guide bundle (not shown), and the like are inserted into the universal cable 8. A connector portion 8a that is connected to the processor 3 is provided at the end portion of the universal cable 8 on the proximal end side. When the connector portion 8a is connected to the processor 3, the signal cable 34 connects electrical components such as an imaging element 31 (see FIG. 4) of the endoscope 2 to the processor 3 such that they can communicate with each other.

As shown in FIG. 4, the optical unit 20 is disposed in the insertion distal end portion 11. The optical unit 20 forms an optical image of the subject. The optical unit 20 has a lens frame 21, a plurality of first lenses 22, and a second lens 23.

The lens frame 21 holds the plurality of first lenses 22 and the second lens 23. The lens frame 21 is disposed in the distal end case 18c. The lens frame 21 has a first lens frame 21a and a second lens frame 21b. The first lens frame 21a has a substantially cylindrical shape centered on the central axis J and extends in the optical axis direction. The outer circumferential surface of the first lens frame 21a is held by the holding portion 18b and the distal end case 18c. The plurality of first lenses 22 are fixed to the inner circumferential surface of the first lens frame 21a. Each of the first lenses 22 is disposed in the optical axis direction.

The second lens frame 21b has a substantially cylindrical shape centered on the central axis J and extends in the optical axis direction. The second lens frame 21b is open on both sides in the optical axis direction. A portion of the second lens frame 21b on the object side (+Z side) is supported in the radial direction by the outer circumferential surface of the first lens frame 21a. The second lens 23 is fixed to the inner circumferential surface of the second lens frame 21b. During assembly of the endoscope 2, the focal position of the optical image of the subject which is formed by the optical unit 20 can be adjusted by moving the second lens frame 21b in the optical axis direction and adjusting the position of the second lens 23 in the optical axis direction. Further, the second lens frame 21b may be movable in the optical axis direction relative to the first lens frame 21a. In this case, during use of the endoscope 2, the position of the second lens 23 in the optical axis direction can be adjusted.

As shown in FIG. 3, a plurality of accommodation objects 70 pass through the inside of the case 18 in the optical axis direction. The accommodation objects 70 are spaced apart from each other in the radial direction or the circumferential direction. In the present embodiment, the plurality of accommodation objects 70 include an air and water supply unit 71, a light guide unit 72, and a treatment tool unit 73. The air and water supply unit 71 is a flow path through which air and liquids such as water are supplied to the outer surface of the first lens 22 that is disposed closest to the object side (+Z side) among the plurality of first lenses 22. By supplying air and liquids to the outer surface of such a first lens 22, body fluids, blood, and the like adhering to the outer surface of the first lens 22 can be removed. The air and water supply unit 71 is connected to a pump (not shown) that pressure-feeds air and liquids.

The light guide unit 72 is an optical path for irradiating the subject with light. The end portion of the light guide unit 72 on the proximal end side is connected to the light guide bundle. The treatment tool unit 73 accommodates a treatment tool such as a forceps, a snare, or a syringe needle, as well as a cable connected to the treatment tool. The cable is connected to the operation section 7.

As shown in FIG. 4, the holding frame 50 has a tubular shape, surrounds the central axis J from the outside in the radial direction, and extends in the optical axis direction. In the present embodiment, the holding frame 50 has a substantially tetragonal tubular shape centered on the central axis J. The holding frame 50 surrounds a portion of the imaging unit 30 on the object side (+Z side) from the outside in the radial direction. The holding frame 50 holds the imaging unit 30. The holding frame 50 has a lens barrel 51 and a unit holding frame 52.

The lens barrel 51 has a substantially tetragonal tubular shape, surrounds the central axis J, and extends in the optical axis direction. The lens barrel 51 is open on both sides in the optical axis direction. The lens barrel 51 surrounds the unit holding frame 52 from the outside in the radial direction. The lens barrel 51 is disposed in the recess 18e of the holding portion 18b. The outer surface of the lens barrel 51 is fixed to the inner surface of the recess 18e. In this way, the holding frame 50 is fixed to the case 18. In the optical axis direction, the position of the end portion of the lens barrel 51 on the proximal end side (−Z side) and the position of the end portion of the holding portion 18b on the proximal end side are substantially the same position.

The unit holding frame 52 has a tubular shape, surrounds the central axis J, and extends in the optical axis direction. The unit holding frame 52 has a tubular shape centered on the central axis J. In the present embodiment, the unit holding frame 52 has a substantially tetragonal tubular shape centered on the central axis J. The unit holding frame 52 is open on both sides in the optical axis direction. The unit holding frame 52 surrounds the imaging unit 30 from the outside in the radial direction. A portion of the unit holding frame 52 on the object side (+Z side) is disposed in the lens barrel 51. A portion of the outer surface of the unit holding frame 52 on the object side is fixed to the inner surface of the lens barrel 51. In this way, the lens barrel 51 holds the unit holding frame 52. A portion of the second lens frame 21b on the proximal end side (−Z side) is inserted into a portion of the inside of the unit holding frame 52 on the object side. A portion of the second lens frame 21b on the proximal end side is held on the inner surface of the unit holding frame 52. In the optical axis direction, the position of the end portion of the unit holding frame 52 on the object side and the position of the end portion of the lens barrel 51 on the object side are substantially the same position. A portion of the unit holding frame 52 on the proximal end side protrudes from the lens barrel 51 toward the proximal end side. The unit holding frame 52 has an opening portion 50a and a facing surface 52a. That is, the holding frame 50 has the opening portion 50a.

The opening portion 50a is the end portion of the unit holding frame 52 on the proximal end side (−Z side). The opening portion 50a is open on the proximal end side. The facing surface 52a is a portion of the outer surface of the unit holding frame 52 which faces the proximal end side. Although not shown in the drawing, the facing surface 52a has a substantially tetragonal ring shape when viewed in the optical axis direction. In the present embodiment, the unit holding frame 52 is made of a metal. In the present embodiment, the unit holding frame 52 is formed by pressing a plate-shaped member.

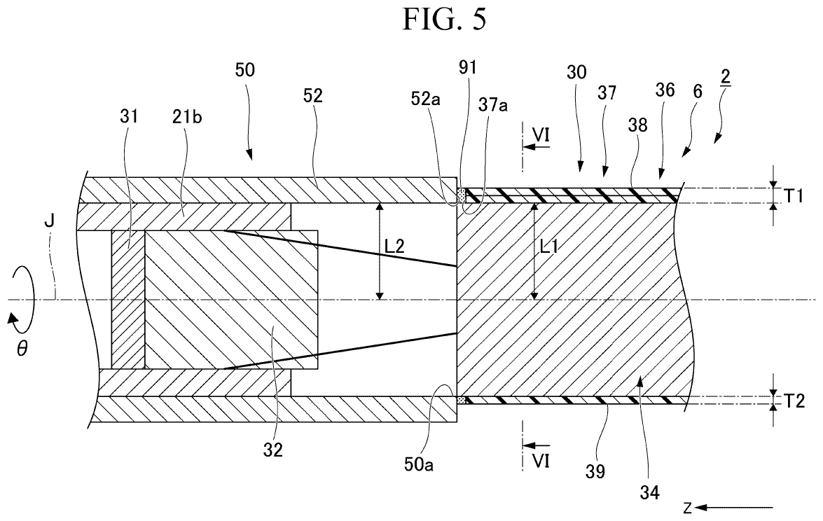

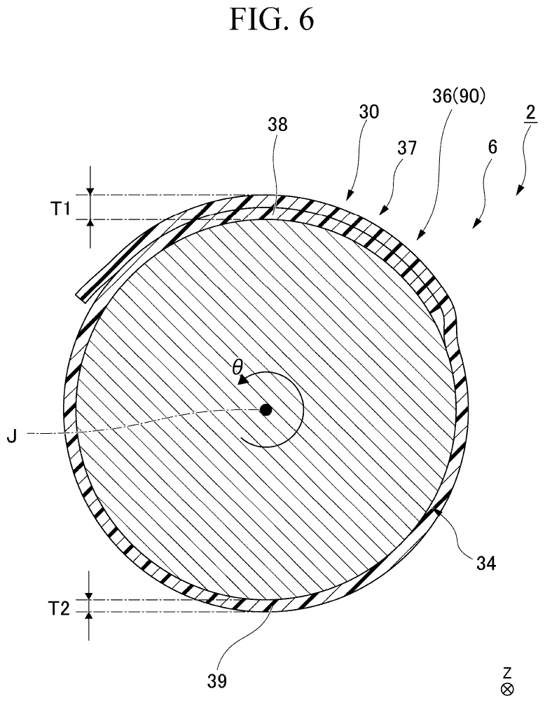

FIG. 5 is a cross-sectional view showing a part of the imaging unit 30 of the present embodiment. FIG. 6 is a cross-sectional view showing a part of the imaging unit 30 of the present embodiment, along line VI-VI in FIG. 5.

As shown in FIG. 4, the imaging unit 30 is disposed in the tubular portion 18a. The imaging unit 30 extends in the tubular portion 18a in the optical axis direction. As shown in FIG. 3, the imaging unit 30 is disposed with a gap with respect to each of the plurality of accommodation objects 70 in the radial direction and the circumferential direction. As shown in FIG. 4, the imaging unit 30 is disposed closer to the proximal end side (−Z side) than the optical unit 20. The imaging unit 30 has an imaging element 31, an imaging board 32, a signal cable 34, and a protective member 36.

The imaging element 31 receives the optical image of the subject which has been formed by the optical unit 20 and converts the optical image into an image signal. The imaging element 31 is, for example, an image sensor such as a CCD or a CMOS. The imaging element 31 is disposed closer to the proximal end side (−Z side) than the optical unit 20. A light receiving surface of the imaging element 31 faces the object side (+Z side). The light receiving surface of the imaging element 31 faces the second lens 23 in the optical axis direction. The imaging element 31 is disposed in the holding frame 50. As shown in FIG. 5, the imaging element 31 is fixed to the inner surface of a portion of the second lens frame 21b on the proximal end side. As described above, the second lens frame 21b is held by the unit holding frame 52. Therefore, the imaging element 31 is held by the holding frame 50 with the second lens frame 21b interposed therebetween. In this way, the holding frame 50 holds the imaging unit 30.

The imaging board 32 is connected to the imaging element 31. The imaging board 32 is, for example, a flexible printed circuit board (FPC board). The imaging board 32 is mounted with electronic components such as a digital IC that generates a drive signal for the imaging element 31 and a capacitor that stabilizes a drive power supply for the digital IC. The imaging board 32 is disposed in the holding frame 50. The imaging board 32 is disposed closer to the proximal end side (−Z side) than the imaging element 31. The signal cable 34 is connected to the imaging board 32. In this way, the imaging board 32 and the processor 3 (see FIG. 1) are connected such that the imaging board 32 and the processor 3 can communicate with each other.

The signal cable 34 connects the imaging board 32 and the processor 3 such that the imaging board 32 and the processor 3 can communicate with each other. The end portion of the signal cable 34 on the proximal end side is connected to the processor 3 shown in FIG. 1. The signal cable 34 passes through the inside of the universal cable 8 and the inside of the rigid tube portion 13. As shown in FIG. 2, a portion of the signal cable 34 on the object side (+Z side) passes through the inside of the bending portion 12 and the inside of the case 18. As shown in FIG. 3, in the case 18, the signal cable 34 is disposed with a gap with respect to each of the plurality of accommodation objects 70 in the radial direction and the circumferential direction. As shown in FIG. 4, the signal cable 34 extends in the optical axis direction. As shown in FIG. 6, the signal cable 34 has a substantially circular shape when viewed in the optical axis direction. As shown in FIG. 4, in the present embodiment, the signal cable 34 is a cable bundle formed by bundling a plurality of cables 34a. The signal cable 34 is inserted into the holding frame 50 through the opening portion 50a. The end portion of each cable 34a on the object side is joined to the imaging board 32 by soldering, for example. In this way, the end portion of the signal cable 34 on the object side is joined to the imaging board 32.

The protective member 36 protects the signal cable 34. The signal cable 34 is fixed to the holding frame 50 by the protective member 36 being fixed to the holding frame 50. The protective member 36 has a tubular shape and extends in the optical axis direction. The protective member 36 has a tubular shape centered on the central axis J. More specifically, the protective member 36 has a substantially cylindrical shape centered on the central axis J. As shown in FIG. 6, the protective member 36 surrounds the signal cable 34 from the outside in the radial direction. In the present embodiment, the protective member 36 is adhesively fixed to the outer circumferential surface of the signal cable 34. The protective member 36 does not have to be fixed to the signal cable 34. As shown in FIG. 5, the protective member 36 has a distal end portion 37.

The distal end portion 37 is a portion of the protective member 36 on the object side including a distal end 37a that is the end portion of the protective member 36 on the object side (+Z side). In the present embodiment, the distal end 37a faces the facing surface 52a of the unit holding frame 52 in the optical axis direction. That is, the distal end 37a faces the holding frame 50 in the optical axis direction. The distal end 37a faces the facing surface 52a closely in the optical axis direction. That is, the protective member 36 faces the holding frame 50 closely in the optical axis direction. As shown in FIG. 6, the distal end portion 37 has a first distal end portion 38 and a second distal end portion 39.

The first distal end portion 38 is a part of the distal end portion 37 in the circumferential direction. The second distal end portion 39 is another part of the distal end portion 37 in the circumferential direction. The first distal end portion 38 and the second distal end portion 39 are connected to each other in the circumferential direction. More specifically, the end portion of the first distal end portion 38 on one side (+θ side) in the circumferential direction is connected to the end portion of the second distal end portion 39 on the other side (−θ side) in the circumferential direction, and the end portion of the first distal end portion 38 on the other side in the circumferential direction is connected to the end portion of the second distal end portion 39 on the one side in the circumferential direction. In the present embodiment, the protective member 36 is made by forming a sheet member 90 having a sheet shape and extending in the optical axis direction into a tubular shape. The first distal end portion 38 is a portion of the distal end portion 37 in which portions of the sheet member 90 overlap each other in the radial direction. In the present embodiment, the first distal end portion 38 is a portion in which portions of the sheet member 90 overlap each other into two layers. The portions of the sheet member 90 that overlap each other in the radial direction are adhesively fixed together with an adhesive (not shown). In this way, the protective member 36 is formed into a tubular shape. The second distal end portion 39 is a portion of the distal end portion 37 at which portions of the sheet member 90 does not overlap each other in the radial direction. For this reason, a thickness T1 of the first distal end portion 38, that is, a thickness of the first distal end portion 38 in the radial direction, is greater than a thickness T2 of the second distal end portion 39, that is, a thickness of the second distal end portion 39 in the radial direction.

The sheet member 90 is made of a resin. In the present embodiment, the sheet member 90 is formed of polytetrafluoroethylene (PTFE). The sheet member 90 may be formed of a resin other than polytetrafluoroethylene. In the present embodiment, the thickness of the sheet member 90 is about 200 μm. The thickness of the sheet member 90 can be appropriately determined depending on the material of the resin forming the sheet member 90, the rigidity required for the protective member 36, and the like.

A first distance L1 shown in FIG. 5 is the minimum distance between the central axis J and the inner surface of the protective member 36 when viewed in the optical axis direction. A second distance L2 is the minimum distance between the central axis J and the inner surface of the holding frame 50 when viewed in the optical axis direction. More specifically, the second distance L2 is the minimum distance between the central axis J and the inner surface of the unit holding frame 52 when viewed in the optical axis direction. In the present embodiment, the first distance L1 is greater than the second distance L2. When viewed in the optical axis direction, the difference between the first distance L1 and the second distance L2 is less than or equal to the thickness T2 of the second distal end portion 39, that is, the thickness of the second distal end portion 39 in the radial direction.

As described above, the distal end 37a of the protective member 36 faces the facing surface 52a of the unit holding frame 52 in the optical axis direction. In the present embodiment, the distal end 37a is adhered to the facing surface 52a with an adhesive 91. That is, the distal end 37a is adhered to the holding frame 50. In other words, the protective member 36 is adhesively fixed to the holding frame 50. In this way, the holding frame 50 holds the imaging unit 30.

According to the present embodiment, the imaging unit 30 has the imaging element 31 that is disposed in the holding frame 50, the imaging board 32 connected to the imaging element 31, the signal cable 34 extending in the optical axis direction, and the protective member 36 having a tubular shape, surrounding the signal cable 34, and extending in the optical axis direction. The distal end 37a of the protective member 36 faces the holding frame 50 in the optical axis direction and is adhered to the holding frame 50, and the distal end portion 37 of the protective member 36, which is a portion on the object side (+Z side) including the distal end 37a, has the first distal end portion 38 and the second distal end portion 39. The first distal end portion 38 and the second distal end portion 39 are connected to each other in the circumferential direction, and the thickness T1 of the first distal end portion 38 is greater than the thickness T2 of the second distal end portion 39. Therefore, the cross-sectional area of the distal end portion 37 can be increased compared to a case in which the distal end portion 37 is formed only by the second distal end portion 39. In this way, the adhering area between the distal end portion 37 and the holding frame 50 can be increased, and thus it is possible to increase the adhering strength between the protective member 36 and the holding frame 50. Therefore, for example, when the imaging unit 30 is removed from the insertion section 6 during maintenance of the endoscope 2, even though a force toward the proximal end side (−Z side) is applied to the signal cable 34, the imaging unit 30 can be prevented from coming off the holding frame 50. Therefore, the imaging unit 30 can be easily removed from the insertion section 6, and thus an increase in the number of steps required for maintenance work on the endoscope 2 can be suppressed.

Further, in the present embodiment, since the protective member 36 faces the holding frame 50 closely in the optical axis direction, the thickness of the adhesive 91 for adhering the protective member 36 and the holding frame 50 to each other can be made to be small. Therefore, the rigidity of the adhesive 91 can be prevented from becoming too high. For this reason, during use of the endoscope 2, even though stress is applied to the protective member 36, the adhesive 91, and the holding frame 50 due to the bending of the signal cable 34 when the bending portion 12 is bent, the adhesive 91 is easily elastically deformed. In this way, it is possible to suppress the concentration of stress on the adhesive 91. Therefore, during use of the endoscope 2, damage to the adhesive 91 can be suppressed, and thus a decrease in the adhering strength between the protective member 36 and the holding frame 50 can be suppressed.

Further, in the present embodiment, the hardness of the adhesive 91 is smaller than the hardness of the holding frame 50 and larger than the hardness of the signal cable 34. Moreover, the signal cable 34, the adhesive 91, and the holding frame 50 are disposed in that order from the proximal end side (−Z side) toward the object side (+Z side). As a result, in the signal cable 34, the adhesive 91, and the holding frame 50, the hardness can be increased in stages from the proximal end side toward the object side. Therefore, even though bending stress is applied to the signal cable 34 when the bending portion 12 is bent, the adhesive 91 and the holding frame 50 are easily elastically deformed in stages, and thus it is possible to prevent the bending stress from concentrating on the signal cable 34. Therefore, it is possible to prevent the signal cable 34 from being damaged.

According to the present embodiment, the imaging board 32 is disposed in the holding frame 50, and the end portion of the signal cable 34 on the object side (+Z side) is joined to the imaging board 32. Therefore, since it is easy to stabilize the connection between the signal cable 34 and the imaging board 32, during assembly of the endoscope 2 or when the imaging unit 30 is removed from the insertion section 6 during maintenance of the endoscope 2, even though a force toward the proximal end side (−Z side) is applied to the signal cable 34, it is easy to prevent the signal cable 34 from coming off the imaging board 32. Further, during use of the endoscope 2, even though bending stress is applied to the signal cable 34 when the bending portion 12 is bent, it is easy to prevent the signal cable 34 from coming off the imaging board 32. As a result, the stability of the operation of the endoscope 2 can be achieved.

According to the present embodiment, the holding frame 50 has the opening portion 50a that is open on the proximal end side (−Z side), and the signal cable 34 is inserted into the holding frame 50 from the opening portion 50a. Therefore, since the signal cable 34 can be joined to the imaging board 32 that is disposed in the holding frame 50 through the opening portion 50a, the work of joining the signal cable 34 and the imaging board 32 can be simplified. Therefore, an increase in the number of steps required for manufacturing the endoscope 2 can be suppressed.

According to the present embodiment, the protective member 36 is made by forming the sheet member 90 having a sheet shape and extending in the optical axis direction into a tubular shape, and the first distal end portion 38 is a portion of the distal end portion 37 in which portions of the sheet member 90 overlap each other in the radial direction. For this reason, the protective member 36 having the first distal end portion 38 can be formed by the simple work of forming the sheet member 90 into a tubular shape. Therefore, an increase in the number of steps required for forming the protective member 36 can be suppressed.

According to the present embodiment, the protective member 36 is adhesively fixed to the signal cable 34. Therefore, since the signal cable 34 is fixed to the holding frame 50 by the protective member 36 being fixed to the holding frame 50, even though a force toward the proximal end side (−Z side) is applied to the signal cable 34 during maintenance of the endoscope 2, the signal cable 34 can be prevented from moving in the optical axis direction. Further, during use of the endoscope 2, even though bending stress is applied to the signal cable 34 when the bending portion 12 is bent, the signal cable 34 can be prevented from moving in the optical axis direction. As a result, during maintenance of the endoscope 2 or use of the endoscope 2, the signal cable 34 can be more suitably prevented from coming off the imaging board 32. Therefore, the stability of the operation of the endoscope 2 can be more suitably achieved.

According to the present embodiment, each of the holding frame 50 and the protective member 36 has a tubular shape centered on the central axis J, and when viewed in the optical axis direction, the difference between the first distance L1, that is, the minimum distance between the central axis J and the inner surface of the protective member 36, and the second distance L2, that is, the minimum distance between the central axis J and the inner surface of the holding frame 50, is less than or equal to the thickness T2 of the second distal end portion 39. Therefore, since it is easy to reduce the difference between the dimension of the holding frame 50 in the radial direction and the dimension of the protective member 36 in the radial direction, it is easy to make the entire distal end 37a face the facing surface 52a in the optical axis direction. Therefore, it is easy to increase the adhering area between the protective member 36 and the holding frame 50. In this way, it is possible to more suitably increase the adhering strength between the protective member 36 and the holding frame 50. For this reason, during maintenance of the endoscope 2, even though a force toward the proximal end side (−Z side) is applied to the signal cable 34, the imaging unit 30 can be more suitably prevented from coming off the holding frame 50. Therefore, an increase in the number of steps required for maintenance work on the endoscope 2 can be more suitably suppressed.

### First Modification Example of First Embodiment

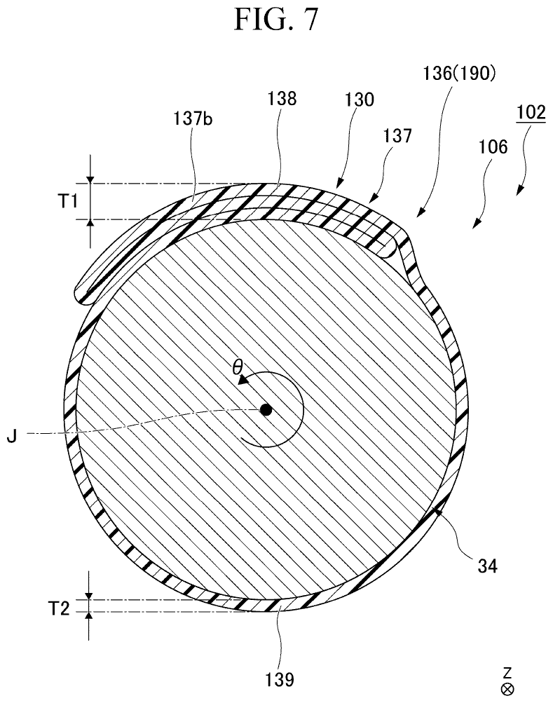

FIG. 7 is a cross-sectional view showing a part of an imaging unit 130 of the present modification example. In the present modification example, an insertion section 106 of an endoscope 102 includes the imaging unit 130. In the following description, the same constituent elements in aspect as those in the first embodiment described above are designated by the same reference signs, and the description thereof will be omitted.

The imaging unit 130 has an imaging element 31, an imaging board 32, a signal cable 34, and a protective member 136. The configurations and the like of the imaging element 31, the imaging board 32, and the signal cable 34 of the present modification example are the same as those of the imaging element 31, the imaging board 32, and the signal cable 34 of the first embodiment described above.

The protective member 136 has a tubular shape and extends in the optical axis direction. In the present modification example, the protective member 136 has a substantially cylindrical shape centered on the central axis J. In the present modification example, the protective member 136 is adhesively fixed to the outer circumferential surface of the signal cable 34. The protective member 136 does not have to be fixed to the signal cable 34. The protective member 136 has a distal end portion 137.

The distal end portion 137 is a portion of the protective member 136 on the object side including a distal end (not shown) that is the end portion of the protective member 136 on the object side (+Z side). The distal end portion 137 has a first distal end portion 138 and a second distal end portion 139. The first distal end portion 138 is a part of the distal end portion 137 in the circumferential direction. The second distal end portion 139 is another part of the distal end portion 137 in the circumferential direction. The first distal end portion 138 and the second distal end portion 139 are connected to each other in the circumferential direction.

In the present modification example, the protective member 136 is constituted by a tubular member 190 having a tubular shape and extending in the optical axis direction. The inner diameter of the tubular member 190 is larger than the outer diameter of the signal cable 34. The protective member 136 is formed by folding a folded portion 137b, which is a part of the tubular member 190 in the circumferential direction, in the circumferential direction, and fixing the folded portion 137b and a portion of the tubular member 190 other than the folded portion 137b to each other. In the present modification example, the folded portion 137b and the portion of the tubular member 190 other than the folded portion 137b are fixed to each other with an adhesive (not shown).

In the present modification example, the first distal end portion 138 is a part of the distal end portion 137 in the circumferential direction and includes the folded portion 137b. In the present modification example, the first distal end portion 138 is a portion in which portions of the tubular member 190 overlap each other into three layers. The second distal end portion 139 is a portion of the distal end portion 137 which does not include the folded portion 137b. For this reason, a thickness T1 of the first distal end portion 138 is greater than a thickness T2 of the second distal end portion 139.

The tubular member 190 is made of a resin. In the present modification example, the tubular member 190 is formed of polytetrafluoroethylene (PTFE). The tubular member 190 may be formed of a resin other than polytetrafluoroethylene. In the present modification example, the thickness of the tubular member 190 is about 200 μm. Other configurations and the like of the imaging unit 130 of the present modification example are the same as those of the imaging unit 30 of the first embodiment described above.

According to the present modification example, the protective member 136 is formed by folding the folded portion 137b, which is a part of the tubular member 190 having a tubular shape and extending in the optical axis direction in the circumferential direction, in the circumferential direction, and fixing the folded portion 137b and the portion other than the folded portion 137b to each other, and the first distal end portion 138 is a portion of the distal end portion 137 which includes the folded portion 137b. For this reason, the protective member 136 having the first distal end portion 138 can be formed by the simple operation work of folding the folded portion 137b of the tubular member 190 in the circumferential direction and fixing the folded portion 137b and the portion other than the folded portion to each other. Therefore, an increase in the number of steps required for forming the protective member 136 can be suppressed.

When the bending portion 12 is bent during use of the endoscope 102, bending stress is applied to the signal cable 34 and each of a plurality of accommodation objects 70. For this reason, the signal cable 34 and each of the plurality of accommodation objects 70 may deform in the circumferential direction and the radial direction and move in the circumferential direction and the radial direction. In this case, the signal cable 34 is pressed against the accommodation objects 70, and thus stress is applied to the signal cable 34. In contrast, in the present modification example, the first distal end portion 138 is a portion of the distal end portion 137 in which portions of the tubular member 190 overlap each other into three layers, and therefore the thickness T1 of the first distal end portion 138 can be easily made large. As a result, even though the signal cable 34 is pressed against the accommodation objects 70, the stress applied to the signal cable 34 can be easily absorbed at the first distal end portion 138, thereby reducing the stress applied to the signal cable 34. Therefore, during use of the endoscope 102, it is easy to prevent the signal cable 34 from being damaged.

Further, in the present modification example, the thickness T1 of the first distal end portion 138 is greater than the thickness T2 of the second distal end portion 139. Therefore, the cross-sectional area of the distal end portion 137 can be increased compared to a case in which the distal end portion 137 is formed only by the second distal end portion 139, and thus the adhering strength between the protective member 136 and a holding frame 50 can be increased. Therefore, as described above, during maintenance of the endoscope 102, even though a force toward the proximal end side (−Z side) is applied to the signal cable 34, the imaging unit 130 can be more suitably prevented from coming off the holding frame 50. Therefore, an increase in the number of steps required for maintenance work on the endoscope 102 can be more suitably suppressed.

### Second Modification Example of First Embodiment

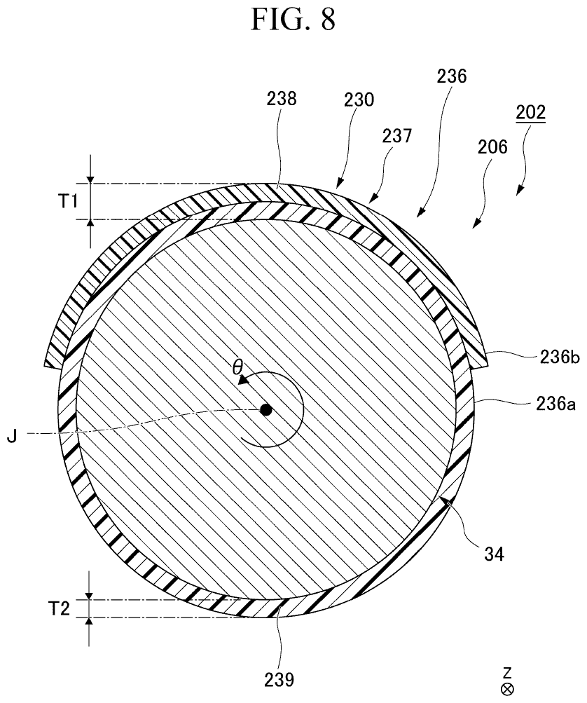

FIG. 8 is a cross-sectional view showing a part of an imaging unit 230 of the present modification example. In the present modification example, an insertion section 206 of an endoscope 202 includes the imaging unit 230. In the following description, the same constituent elements in aspect as those in the first embodiment described above are designated by the same reference signs, and the description thereof will be omitted.

The imaging unit 230 has an imaging element 31, an imaging board 32, a signal cable 34, and a protective member 236. The configurations and the like of the imaging element 31, the imaging board 32, and the signal cable 34 of the present modification example are the same as those of the imaging element 31, the imaging board 32, and the signal cable 34 of the first embodiment described above.

The protective member 236 has a tubular shape and extends in the optical axis direction. In the present modification example, the protective member 236 has a substantially cylindrical shape centered on the central axis J. The protective member 236 is adhesively fixed to the outer circumferential surface of the signal cable 34. The protective member 236 does not have to be fixed to the signal cable 34. The protective member 236 has a first protective member 236a, a second protective member 236b, and a distal end portion 237.

The first protective member 236a has a tubular shape and extends in the optical axis direction. In the present modification example, the first protective member 236a has a substantially cylindrical shape centered on the central axis J. The first protective member 236a surrounds the signal cable 34 from the outside in the radial direction.

The second protective member 236b extends in the optical axis direction. When viewed in the optical axis direction, the second protective member 236b has a substantially arcuate shape centered on the central axis J. The second protective member 236b is disposed outside the first protective member 236a in the radial direction. The second protective member 236b is in contact with a part of the outer circumferential surface of the first protective member 236a in the circumferential direction. The second protective member 236b may be fixed or may not be fixed to a part of the outer circumferential surface of the first protective member 236a in the circumferential direction. The first protective member 236a and the second protective member 236b are made of a resin. In the present modification example, the first protective member 236a and the second protective member 236b are formed of polytetrafluoroethylene (PTFE). In the present modification example, each of the thickness of the first protective member 236a and the thickness of the second protective member 236b is about 200 μm.

The distal end portion 237 is a portion of the protective member 236 on the object side including a distal end (not shown) that is the end portion of the protective member 236 on the object side (+Z side). The distal end portion 237 has a first distal end portion 238 and a second distal end portion 239. The first distal end portion 238 is a part of the distal end portion 237 in the circumferential direction. The second distal end portion 239 is another part of the distal end portion 237 in the circumferential direction. The first distal end portion 238 and the second distal end portion 239 are connected to each other in the circumferential direction.

In the present modification example, the first distal end portion 238 is constituted by a portion of the first protective member 236a which overlaps the second protective member 236b in the radial direction, and the second protective member 236b. In the present modification example, the second distal end portion 239 is constituted by a portion of the first protective member 236a which does not overlap the second protective member 236b in the radial direction. A thickness T1 of the first distal end portion 238 is greater than a thickness T2 of the second distal end portion 239. Other configurations and the like of the imaging unit 230 of the present modification example are the same as those of the imaging unit 30 of the first embodiment described above.

According to the present modification example, the protective member 236 has the first protective member 236a having a tubular shape, surrounding the signal cable 34, and extending in the optical axis direction, and the second protective member 236b in contact with a part of the outer circumferential surface of the first protective member 236a in the circumferential direction, and the first distal end portion 238 is constituted by the portion of the first protective member 236a which overlaps the second protective member 236b in the radial direction, and the second protective member 236b. For this reason, the first distal end portion 238 can be formed by a simple configuration in which the second protective member 236b is disposed outside the first protective member 236a in the radial direction, and therefore the protective member 236 can be easily formed. Therefore, an increase in the number of steps required for forming the protective member 236 can be suppressed.

### Third Modification Example of First Embodiment

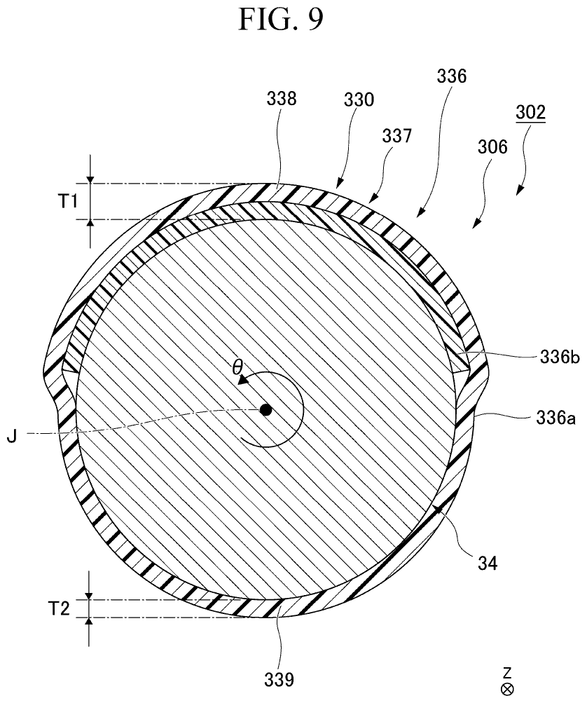

FIG. 9 is a cross-sectional view showing a part of an imaging unit 330 of the present modification example. In the present modification example, an insertion section 306 of an endoscope 302 includes the imaging unit 330. In the following description, the same constituent elements in aspect as those in the first embodiment described above are designated by the same reference signs, and the description thereof will be omitted.

The imaging unit 330 has an imaging element 31, an imaging board 32, a signal cable 34, and a protective member 336. The configurations and the like of the imaging element 31, the imaging board 32, and the signal cable 34 of the present modification example are the same as those of the imaging element 31, the imaging board 32, and the signal cable 34 of the first embodiment described above.

The protective member 336 has a tubular shape and extends in the optical axis direction. The protective member 336 is adhesively fixed to the outer circumferential surface of the signal cable 34. The protective member 336 has a first protective member 336a, a second protective member 336b, and a distal end portion 337.

The first protective member 336a has a tubular shape and extends in the optical axis direction. In the present modification example, the first protective member 336a has a substantially cylindrical shape centered on the central axis J. The first protective member 336a surrounds the signal cable 34 from the outside in the radial direction.

The second protective member 336b extends in the optical axis direction. When viewed in the optical axis direction, the second protective member 336b has a substantially arcuate shape centered on the central axis J. The second protective member 336b is disposed between the signal cable 34 and the first protective member 336a in the radial direction. The second protective member 336b is in contact with a part of the inner circumferential surface of the first protective member 336a in the circumferential direction. The second protective member 336b may be fixed or may not be fixed to a part of the inner circumferential surface of the first protective member 336a in the circumferential direction. The first protective member 336a and the second protective member 336b are made of a resin. In the present modification example, each of the thickness of the first protective member 336a and the thickness of the second protective member 336b is about 200 μm.

The distal end portion 337 is a portion of the protective member 336 on the object side including a distal end (not shown) that is the end portion of the protective member 336 on the object side (+Z side). The distal end portion 337 has a first distal end portion 338 and a second distal end portion 339. The first distal end portion 338 is a part of the distal end portion 337 in the circumferential direction. The second distal end portion 339 is another part of the distal end portion 337 in the circumferential direction. The first distal end portion 338 and the second distal end portion 339 are connected to each other in the circumferential direction.

In the present modification example, the first distal end portion 338 is constituted by a portion of the first protective member 336a which overlaps the second protective member 336b in the radial direction, and the second protective member 336b. In the present modification example, the second distal end portion 339 is constituted by a portion of the first protective member 336a which does not overlap the second protective member 336b in the radial direction. A thickness T1 of the first distal end portion 338 is greater than a thickness T2 of the second distal end portion 339. Other configurations and the like of the imaging unit 330 of the present modification example are the same as those of the imaging unit 230 of the second modification example of first described above.

According to the present modification example, the protective member 336 has the first protective member 336a having a tubular shape, surrounding the signal cable 34, and extending in the optical axis direction, and the second protective member 336b in contact with a part of the outer circumferential surface of the first protective member 336a in the circumferential direction, and the first distal end portion 338 is constituted by a portion of the first protective member 336a which overlaps the second protective member 336b in the radial direction, and the second protective member 336b. For this reason, the first distal end portion 338 can be formed by a simple configuration in which the second protective member 336b is disposed inside the first protective member 336a in the radial direction, and therefore the protective member 336 can be easily formed. Therefore, an increase in the number of steps required for forming the protective member 336 can be suppressed.

### Fourth Modification Example of First Embodiment

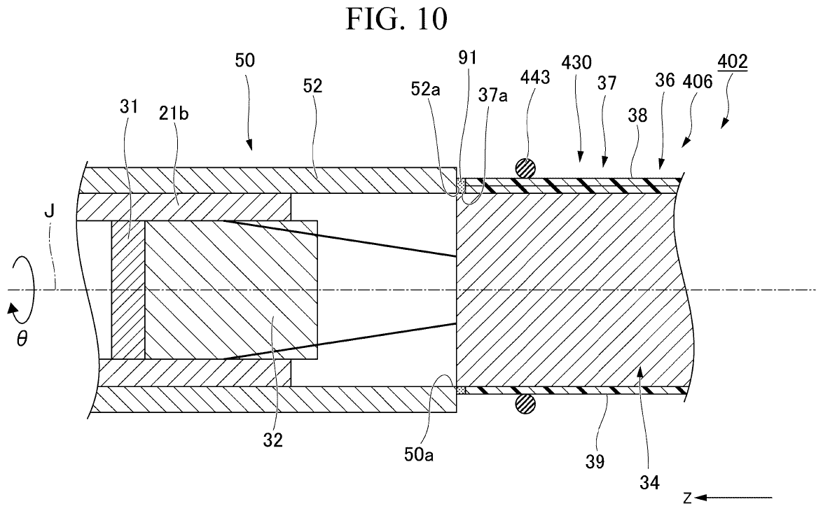

FIG. 10 is a cross-sectional view showing a part of an imaging unit 430 of the present modification example. In the present modification example, an insertion section 406 of an endoscope 402 includes the imaging unit 430. In the following description, the same constituent elements in aspect as those in the first embodiment described above are designated by the same reference signs, and the description thereof will be omitted.

The imaging unit 430 has an imaging element 31, an imaging board 32, a signal cable 34, a protective member 36, and a binding member 443. The configurations and the like of the imaging element 31, the imaging board 32, the signal cable 34, and the protective member 36 of the present modification example are the same as those of the imaging element 31, the imaging board 32, the signal cable 34, and the protective member 36 of the first embodiment described above.

The binding member 443 has a string shape and extends in the circumferential direction. The binding member 443 surrounds the protective member 36 from the outside in the radial direction. The binding member 443 is bound to the protective member 36. The protective member 36 and the signal cable 34 are fixed to each other by the binding member 443. In the present modification example, the binding member 443 is formed of a synthetic fiber such as nylon. Other configurations and the like of the imaging unit 430 of the present modification example are the same as those of the imaging unit 30 of the first embodiment described above.

According to the present modification example, the imaging unit 430 has the binding member 443 that is bound to the protective member 36, and the protective member 36 and the signal cable 34 are fixed to each other by the binding member 443. Therefore, during assembly of the endoscope 2 or during maintenance of the endoscope 2, even though a force toward the proximal end side (−Z side) is applied to the signal cable 34, the signal cable 34 can be prevented from moving in the optical axis direction. As a result, during assembly of the endoscope 2 or during maintenance of the endoscope 2, the signal cable 34 can be more suitably prevented from coming off the imaging board 32.

### Fifth Modification Example of First Embodiment

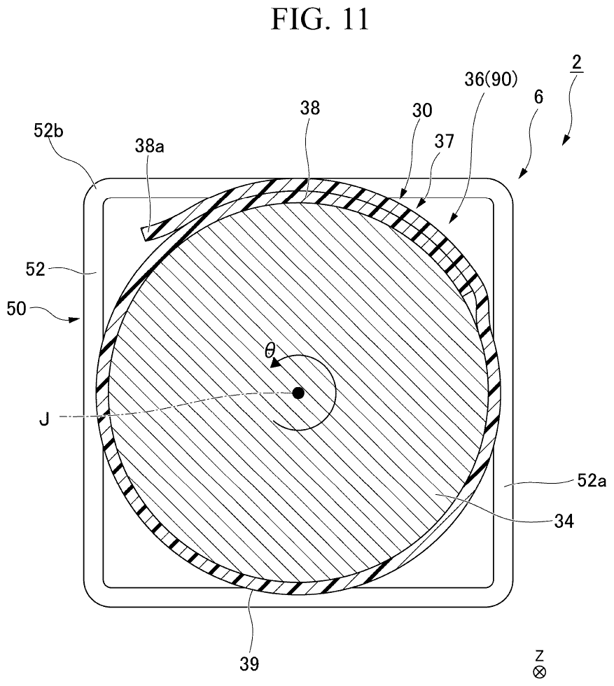

FIG. 11 is a cross-sectional view showing a part of an imaging unit 30 of the present modification example. In the following description, the same constituent elements in aspect as those in the first embodiment described above are designated by the same reference signs, and the description thereof will be omitted.

As described above, a protective member 36 is made by forming a sheet member 90 having a sheet shape and extending in the optical axis direction into a tubular shape. More specifically, the sheet member 90 is formed by being wrapped around the signal cable 34 toward one side (+θ) in the circumferential direction. In addition, as described above, a unit holding frame 52 has a substantially tetragonal tubular shape centered on the central axis J. In the present modification example, one end portion 38a, which is an end portion on one side of the sheet member 90 in the circumferential direction, overlaps a corner portion 52b of the unit holding frame 52 when viewed in the radial direction. The corner portion 52b is a portion of the unit holding frame 52 which is the farthest from the central axis J in the radial direction. Therefore, in the present modification example, even though the one end portion 38a of the sheet member 90 comes off the sheet member 90 outward in the radial direction, it is easy to prevent the one end portion 38a from protruding outward from the unit holding frame 52. Therefore, it is easy to prevent the protective member 36 from becoming larger in the radial direction than a holding frame 50.

### Sixth Modification Example of First Embodiment

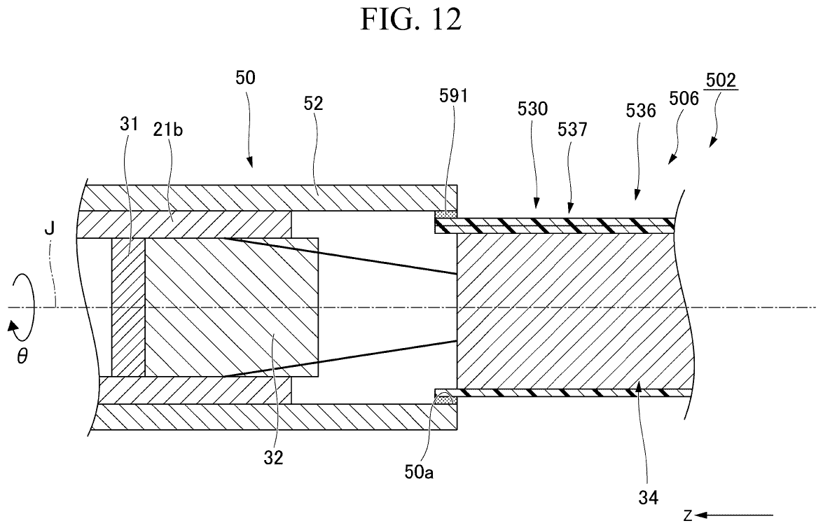

FIG. 12 is a cross-sectional view showing a part of an imaging unit 530 of the present modification example. In the present modification example, an insertion section 506 of an endoscope 502 includes the imaging unit 530. In the following description, the same constituent elements in aspect as those in the first embodiment described above are designated by the same reference signs, and the description thereof will be omitted.

The imaging unit 530 has an imaging element 31, an imaging board 32, a signal cable 34, and a protective member 536. The configurations and the like of the imaging element 31, the imaging board 32, and the signal cable 34 of the present modification example are the same as those of the imaging element 31, the imaging board 32, and the signal cable 34 of the first embodiment described above.

The protective member 536 has a tubular shape and extends in the optical axis direction. The protective member 536 is adhesively fixed to the outer circumferential surface of the signal cable 34. The outer diameter of the protective member 536 of the present modification example is smaller than the outer diameter of the protective member 36 of the first embodiment described above. Other configurations and the like of the protective member 536 of the present modification example are the same as those of the protective member 36 of the first embodiment described above.

In the present modification example, a portion of a distal end portion 537 of the protective member 536 on the object side (+Z side) is inserted into a unit holding frame 52. That is, the portion of the distal end portion 537 of the protective member 536 on the object side is inserted into a holding frame 50. The outer surface of the protective member 536 is fixed to the inner surface of the unit holding frame 52 with an adhesive 591. In this way, the protective member 536 is fixed to the inner surface of the holding frame 50. The outer surface of the protective member 536 may be adhesively fixed to the unit holding frame 52 over the entire circumference in the circumferential direction, or only a part of the outer surface in the circumferential direction may be adhesively fixed to the unit holding frame 52. Other configurations and the like of the imaging unit 530 of the present modification example are the same as those of the imaging unit 30 of the first embodiment described above.

According to the present modification example, the outer surface of the protective member 536 is adhesively fixed to the inner surface of the holding frame 50, and therefore it is easy to increase the adhering area between the protective member 536 and the holding frame 50. Therefore, it is possible to increase the adhering strength between the protective member 536 and the holding frame 50.

### Second Embodiment

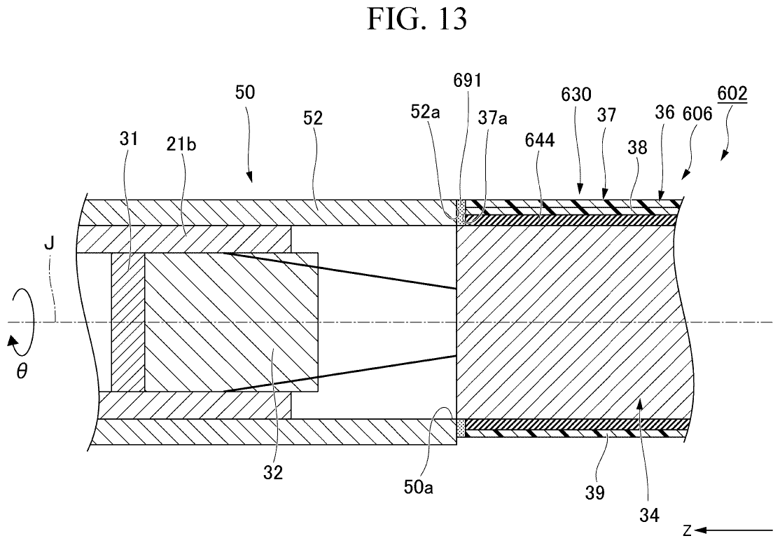

FIG. 13 is a cross-sectional view showing a part of an imaging unit 630 of the present embodiment. In the present embodiment, an insertion section 606 of an endoscope 602 includes the imaging unit 630. In the following description, the same constituent elements in aspect as those in the first embodiment described above are designated by the same reference signs, and the description thereof will be omitted.

The imaging unit 630 has an imaging element 31, an imaging board 32, a signal cable 34, a protective member 36, and an elastic member 644. The configurations and the like of the imaging element 31, the imaging board 32, the signal cable 34, and the protective member 36 of the present embodiment are the same as those of the imaging element 31, the imaging board 32, the signal cable 34, and the protective member 36 of the first embodiment described above.

The elastic member 644 has a tubular shape and extends in the optical axis direction. In the present embodiment, the elastic member 644 has a substantially cylindrical shape centered on the central axis J. The elastic member 644 is disposed between a distal end portion 37 of the protective member 36 and the signal cable 34. In the present embodiment, the elastic member 644 is fixed to each of the protective member 36 and the signal cable 34. The elastic member 644 does not have to be fixed to at least one of the protective member 36 and the signal cable 34. An end portion of the elastic member 644 on the object side (+Z side) faces a facing surface 52a of a unit holding frame 52 in the optical axis direction. In the present embodiment, each of the end portion of the elastic member 644 on the object side and a distal end 37a of the protective member 36 is fixed to the facing surface 52a with an adhesive 691. As a result, each of the elastic member 644 and the protective member 36 is fixed to a holding frame 50. The elastic member 644 does not have to be fixed to the holding frame 50. In the present embodiment, the elastic member 644 is formed of a rubber material. Other configurations and the like of the imaging unit 630 of the present embodiment are the same as those of the imaging unit 30 of the first embodiment described above.

According to the present embodiment, the imaging unit 630 has the elastic member 644 that is disposed between the distal end portion 37 and the signal cable 34. For this reason, in the signal cable 34, the distal end portion 37, the elastic member 644, the adhesive 691, and the holding frame 50, a change in rigidity from the proximal end side (−Z side) toward the object side (+Z side) can be made more gradual. Therefore, even though bending stress is applied to the signal cable 34 when a bending portion 12 is bent, the distal end portion 37, the elastic member 644, the adhesive 691, and the holding frame 50 are elastically deformed in stages, and thus it is possible to more suitably prevent the bending stress from concentrating on the signal cable 34. Therefore, it is possible to more suitably prevent the signal cable 34 from being damaged.

Further, in the present embodiment, each of the elastic member 644 and the protective member 36 is fixed to a holding frame 50. Therefore, since it is easy to fix at least one of the elastic member 644 and the protective member 36 to the holding frame 50 over the entire circumference in the circumferential direction, it is easy to reduce variation in adhering strength between the elastic member 644 or the protective member 36 and the holding frame 50 in the circumferential direction. Therefore, during assembly of the endoscope 602 or during maintenance of the endoscope 602, even though a force toward the proximal end side (−Z side) is applied to the signal cable 34, the imaging unit 630 can be more suitably prevented from coming off the holding frame 50.

### Modification Example of Second Embodiment

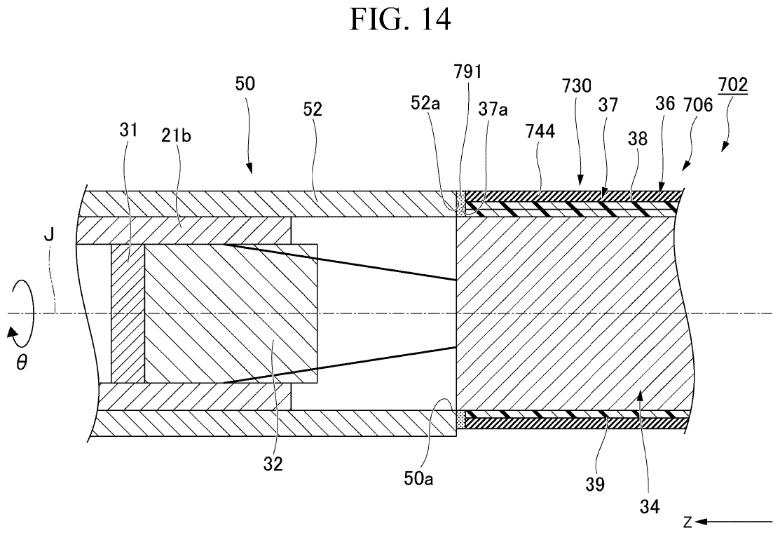

FIG. 14 is a cross-sectional view showing a part of an imaging unit 730 of the present embodiment. In the present modification example, an insertion section 706 of an endoscope 702 includes the imaging unit 730. In the following description, the same constituent elements in aspect as those in the second embodiment described above are designated by the same reference signs, and the description thereof will be omitted.

The imaging unit 730 has an imaging element 31, an imaging board 32, a signal cable 34, a protective member 36, and an elastic member 744. The configurations and the like of the imaging element 31, the imaging board 32, the signal cable 34, and the protective member 36 of the present modification example are the same as those of the imaging element 31, the imaging board 32, the signal cable 34, and the protective member 36 of the second embodiment described above.

The elastic member 744 has a tubular shape and extends in the optical axis direction. In the present modification example, the elastic member 744 has a substantially cylindrical shape centered on the central axis J. The elastic member 744 covers a distal end portion 37 of the protective member 36 from the outside in the radial direction. In the present modification example, the elastic member 744 is fixed to the protective member 36. In the present modification example, each of the end portion of the elastic member 744 on the object side (+Z side) and a distal end 37a of the protective member 36 is fixed to a facing surface 52a with an adhesive 791. As a result, each of the elastic member 744 and the protective member 36 is fixed to a holding frame 50. In the present modification example, the elastic member 744 is formed of a rubber material.

Other configurations and the like of the imaging unit 730 of the present modification example are the same as those of the imaging unit 630 of the second embodiment described above.

According to the present modification example, the imaging unit 730 has the elastic member 744 that covers the distal end portion 37 from the outside in the radial direction. For this reason, in the signal cable 34, the distal end portion 37, the elastic member 744, the adhesive 691, and the holding frame 50, a change in rigidity from the proximal end side (−Z side) toward the object side (+Z side) can be made more gradual. Therefore, even though bending stress is applied to the signal cable 34 when a bending portion 12 is bent, the distal end portion 37, the elastic member 744, the adhesive 791, and the holding frame 50 are elastically deformed in stages, and thus it is possible to more suitably prevent the bending stress from concentrating on the signal cable 34. Therefore, it is possible to more suitably prevent the signal cable 34 from being damaged.

Further, in the present modification example, each of the elastic member 744 and the protective member 36 is fixed to the holding frame 50. Therefore, as in the second embodiment described above, it is easy to reduce variation in adhering strength between the elastic member 744 or the protective member 36 and the holding frame 50 in the circumferential direction. Therefore, during assembly of the endoscope 702 or during maintenance of the endoscope 702, even though a force toward the proximal end side (−Z side) is applied to the signal cable 34, the imaging unit 730 can be more suitably prevented from coming off the holding frame 50.

### Third Embodiment

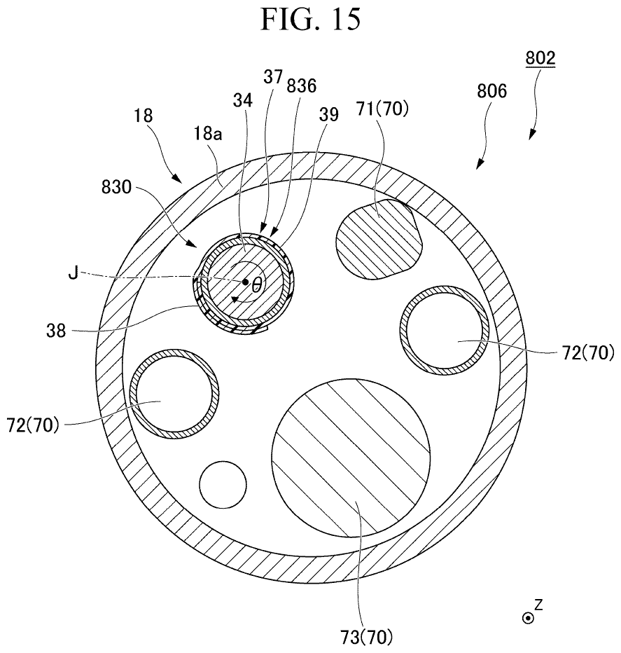

FIG. 15 is a cross-sectional view showing an insertion section 806 of the present embodiment. In the present embodiment, the insertion section 806 of an endoscope 802 includes an imaging unit 830. In the following description, the same constituent elements in aspect as those in the first embodiment described above are designated by the same reference signs, and the description thereof will be omitted.

In the present embodiment, a first distal end portion 38 which is a part of a distal end portion 37 of a protective member 836 in the circumferential direction faces a light guide unit 72 in the radial direction. That is, the first distal end portion 38 faces at least one of a plurality of accommodation objects 70 in the radial direction. The first distal end portion 38 may face either an air and water supply unit 71 or a treatment tool unit 73 in the radial direction. It is preferable that the first distal end portion 38 radially face the accommodation object 70 that has the shortest radial distance from a signal cable 34 among the plurality of accommodation objects 70. Other configurations and the like of the insertion section 806 of the present embodiment are the same as those of the insertion section 6 of the first embodiment described above.

According to the present embodiment, the first distal end portion 38 faces at least one of the plurality of accommodation objects 70 in the radial direction. As described above, during use of the endoscope 802, when the bending portion 12 is bent, the protective member 836 and the signal cable 34 may be pressed against the accommodation object 70, causing stress to be applied to the signal cable 34 with the protective member 836 interposed therebetween. Further, as described above, a thickness T1 of the first distal end portion 38 is greater than a thickness T2 of a second distal end portion 39. In the present embodiment, when the bending portion 12 is bent, the signal cable 34 is pressed against the accommodation object 70 with the first distal end portion 38 interposed therebetween, and thus the stress applied to the signal cable 34 is easily absorbed by the protective member 836 compared to a case in which the signal cable 34 is pressed against the accommodation object 70 with the second distal end portion 39 interposed therebetween. As a result, it is easy to reduce the stress applied to the signal cable 34, and thus the signal cable 34 can be prevented from being damaged during use of the endoscope 802.

### Modification Example of Third Embodiment

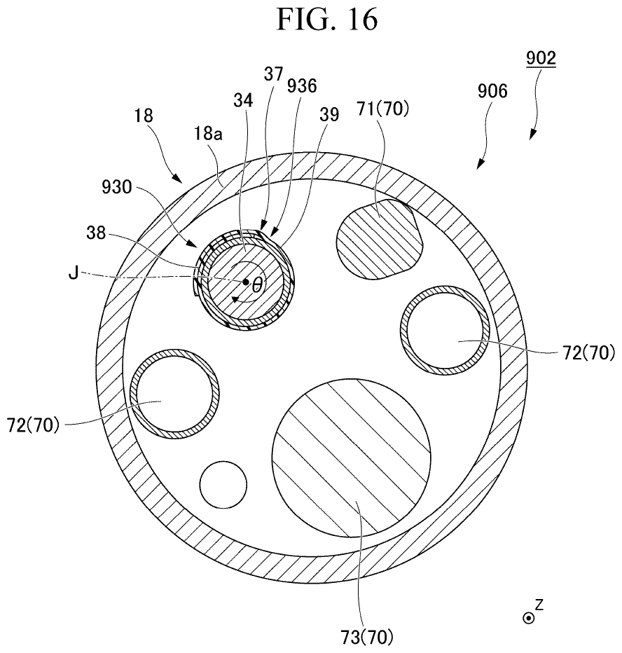

FIG. 16 is a cross-sectional view showing an insertion section 906 of the present modification example. In the present modification example, the insertion section 906 of an endoscope 902 includes an imaging unit 930. In the following description, the same constituent elements in aspect as those in the first embodiment described above are designated by the same reference signs, and the description thereof will be omitted.

In the present modification example, a first distal end portion 38 which is a part of a distal end portion 37 of a protective member 936 in the circumferential direction faces a tubular portion 18a in the radial direction. That is, the first distal end portion 38 faces a case 18 in the radial direction. Other configurations and the like of the insertion section 906 of the present modification example are the same as those of the insertion section 6 of the first embodiment described above.

According to the present modification example, the first distal end portion 38 faces the case 18 in the radial direction. When the insertion section 906 is gripped by an operator during assembly or the like of the endoscope 902, a force toward the inside in the radial direction is applied from the case 18 to the imaging unit 930 and each of the accommodation objects 70. Since the force applied to each of the accommodation objects 70 is absorbed by elastic deformation or the like of each of the accommodation objects 70, the stress applied to the imaging unit 930 via each of the accommodation objects 70 is small. On the other hand, the stress directly applied from the case 18 to the imaging unit 930 is large. In the present modification example, as described above, the first distal end portion 38, which is thicker than a second distal end portion 39, faces the case 18 in the radial direction, and thus the stress applied to the signal cable 34 is easily absorbed by the protective member 936 compared to a case in which the second distal end portion 39 faces the case 18. As a result, it is easy to reduce the stress applied to the signal cable 34, and thus the signal cable 34 can be prevented from being damaged during assembly or the like of the endoscope 902.

### Fourth Embodiment

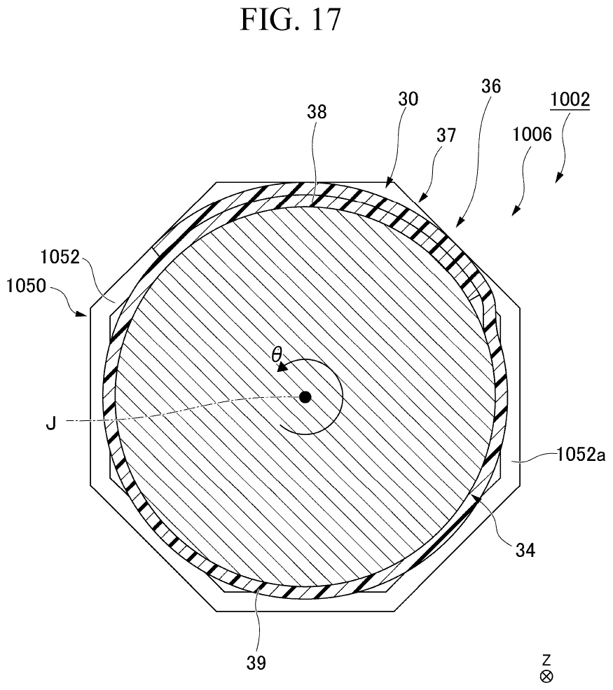

FIG. 17 is a cross-sectional view showing an insertion section 1006 of the present embodiment. In the present embodiment, the insertion section 1006 of an endoscope 1002 includes a holding frame 1050. In the following description, the same constituent elements in aspect as those in the first embodiment described above are designated by the same reference signs, and the description thereof will be omitted.

The holding frame 1050 has a lens barrel (not shown) and a unit holding frame 1052. In the present embodiment, the lens barrel has an octagonal tubular shape centered on the central axis J and extends in the optical axis direction. When viewed in the optical axis direction, the lens barrel has an octagonal ring shape.

In the present embodiment, the unit holding frame 1052 has an octagonal tubular shape centered on the central axis J and extends in the optical axis direction. In the present embodiment, the holding frame 1050 has an octagonal ring shape when viewed in the optical axis direction. Although not shown, a portion of the outer surface of the unit holding frame 1052 on the object side (+Z side) is fixed to the inner surface of the lens barrel (not shown). In this way, the lens barrel holds the unit holding frame 1052. The unit holding frame 1052 has a facing surface 1052a. The facing surface 1052a is a portion of the outer surface of the unit holding frame 1052 which faces the proximal end side (−Z side). When viewed in the optical axis direction, the facing surface 1052a has an octagonal ring shape. In the present embodiment, the unit holding frame 1052 is made of a metal. In the present embodiment, the unit holding frame 1052 is formed by pressing a plate-shaped member. Other configurations and the like of the holding frame 1050 of the present embodiment are the same as those of the holding frame 50 of the first embodiment described above.

As described above, a protective member 36 of the present embodiment has a substantially cylindrical shape centered on the central axis J. When viewed in the optical axis direction, the protective member 36 has a circular ring shape. Although not shown in the figure, the distal end of the protective member 36 faces the facing surface 1052a in the optical axis direction. The distal end of the protective member 36 is fixed to the facing surface 1052a with an adhesive.

According to the present embodiment, when viewed in the optical axis direction, the protective member 36 has a circular ring shape and the holding frame 1050 has an octagonal ring shape. Therefore, when viewed in the optical axis direction, the shape of the unit holding frame 1052 can be made close to the shape of the protective member 36 compared to a case in which the unit holding frame 1052 is, for example, a tetragonal or hexagonal ring shape. As a result, it is easy to more suitably suppress variation in contact area between the protective member 36 and the holding frame 1050 in the circumferential direction, and therefore it is easy to more suitably reduce variation in adhering strength between the protective member 36 and the holding frame 1050 in the circumferential direction. Therefore, during assembly of the endoscope 1002 or during maintenance of the endoscope 1002, even though a force toward the proximal end side (−Z side) is applied to the signal cable 34, the imaging unit 30 can be more suitably prevented from coming off the holding frame 1050.

### Modification Example of Fourth Embodiment

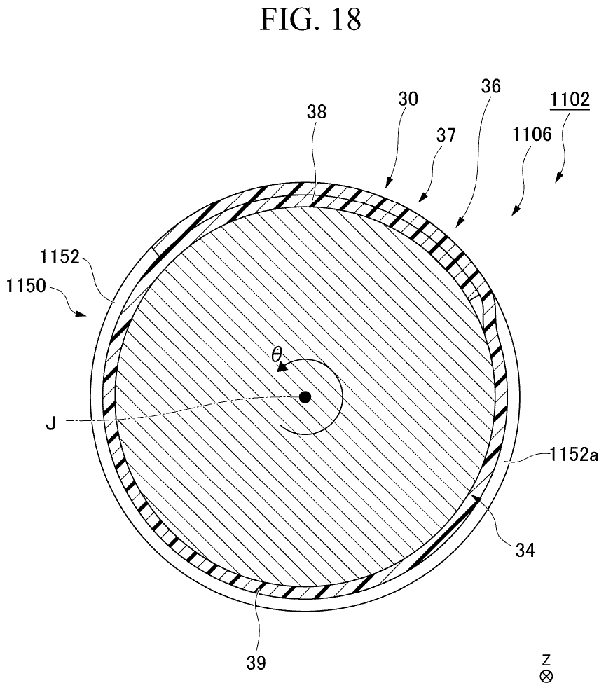

FIG. 18 is a cross-sectional view showing an insertion section 1106 of the present modification example. In the present modification example, the insertion section 1106 of an endoscope 1102 includes a holding frame 1150. In the following description, the same constituent elements in aspect as those in the fourth embodiment described above are designated by the same reference signs, and the description thereof will be omitted.

The holding frame 1150 has a lens barrel (not shown) and a unit holding frame 1152. In the present modification example, the lens barrel has a cylindrical shape centered on the central axis J and extends in the optical axis direction. When viewed in the optical axis direction, the lens barrel has a circular ring shape.

In the present modification example, the unit holding frame 1152 has a cylindrical shape centered on the central axis J and extends in the optical axis direction. In the present modification example, the holding frame 1150 has a circular ring shape when viewed in the optical axis direction. The unit holding frame 1152 has a facing surface 1152a. The facing surface 1152a is a portion of the outer surface of the unit holding frame 1152 which faces the proximal end side (−Z side). When viewed in the optical axis direction, the facing surface 1152a has a circular ring shape. In the present modification example, the unit holding frame 1152 is made of a metal. Other configurations and the like of the holding frame 1150 of the present modification example are the same as those of the holding frame 1050 of the fourth embodiment described above.

As described above, a protective member 36 of the present modification example has a substantially cylindrical shape centered on the central axis J. When viewed in the optical axis direction, the protective member 36 has a circular ring shape. Although not shown in the figure, the distal end of the protective member 36 faces the facing surface 1152a in the optical axis direction. The distal end of the protective member 36 is fixed to the facing surface 1152a with an adhesive.

According to the present modification example, when viewed in the optical axis direction, each of the protective member 36 and the holding frame 1150 has a circular ring shape. Therefore, when viewed in the optical axis direction, the shape of the unit holding frame 1152 can be made closer to the shape of the protective member 36 than a case in which the unit holding frame 1152 is, for example, a tetragonal or hexagonal ring shape. As a result, it is easy to more suitably suppress variation in contact area between the protective member 36 and the holding frame 1150 in the circumferential direction, and therefore it is easy to more suitably reduce variation in adhering strength between the protective member 36 and the holding frame 1150 in the circumferential direction. Therefore, during assembly of the endoscope 1102 or during maintenance of the endoscope 1102, even though a force toward the proximal end side (−Z side) is applied to the signal cable 34, the imaging unit 30 can be more suitably prevented from coming off the holding frame 1150.

### Fifth Embodiment

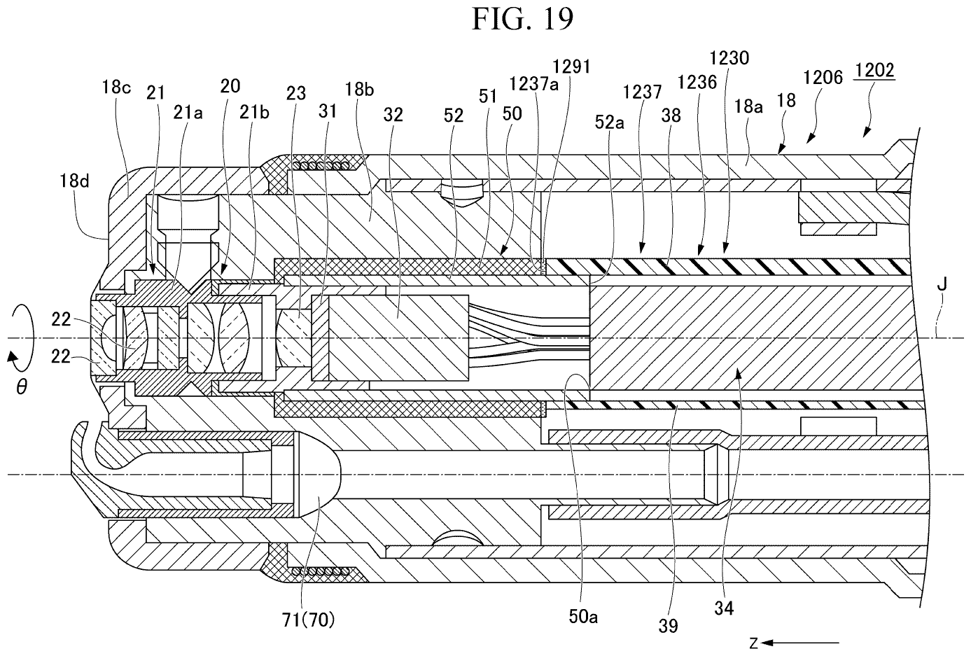

FIG. 19 is a cross-sectional view showing an insertion section 1206 of the present embodiment. In the present embodiment, the insertion section 1206 of an endoscope 1202 includes an imaging unit 1230. In the following description, the same constituent elements in aspect as those in the first embodiment described above are designated by the same reference signs, and the description thereof will be omitted.

The imaging unit 1230 has an imaging element 31, an imaging board 32, a signal cable 34, and a protective member 1236. The configurations and the like of the imaging element 31, the imaging board 32, and the signal cable 34 of the present embodiment are the same as those of the imaging element 31, the imaging board 32, and the signal cable 34 of the first embodiment described above.

The protective member 1236 protects the signal cable 34. The protective member 1236 has a tubular shape and extends in the optical axis direction. In the present embodiment, the protective member 1236 has a substantially cylindrical shape centered on the central axis J. The protective member 1236 has a distal end portion 1237.

The distal end portion 1237 is a portion of the protective member 1236 on the object side including a distal end 1237a that is the end portion of the protective member 1236 on the object side (+Z side). The distal end portion 1237 is disposed outside a unit holding frame 52 in the radial direction. In the present embodiment, the distal end 1237a faces the end portion of a lens barrel 51 on the proximal end side (−Z side) in the optical axis direction. That is, the distal end 1237a faces a holding frame 50 in the optical axis direction. The distal end 1237a is adhered to the end portion of the lens barrel 51 on the proximal end side (−Z side) with an adhesive 1291. In this way, the distal end 1237a is adhered to the lens barrel 51. Therefore, the protective member 1236 is adhesively fixed to the holding frame 50. In this way, the holding frame 50 holds the imaging unit 1230. As described above, the lens barrel 51 surrounds the unit holding frame 52 from the outside in the radial direction. Therefore, the dimension of the lens barrel 51 in the radial direction is greater than the dimension of the unit holding frame 52 in the radial direction. For this reason, the area of the surface of the lens barrel 51 facing the proximal end side easily becomes larger than the area of a facing surface 52a of the unit holding frame 52. In the present embodiment, the area of the surface of the lens barrel 51 facing the proximal end side is larger than the area of the facing surface 52a of the unit holding frame 52.

According to the present embodiment, the distal end 1237a of the protective member 1236 faces the lens barrel 51 in the optical axis direction and is adhered to the lens barrel 51. As described above, the area of the surface of the lens barrel 51 facing the proximal end side (−Z side) is larger than the area of the facing surface 52a of the unit holding frame 52. For this reason, compared to the case in which the protective member 1236 is adhered to the unit holding frame 52, in the present embodiment, it is easy to increase the adhering area between the protective member 1236 and the holding frame 50. Therefore, it is possible to more suitably increase the adhering strength between the protective member 1236 and the holding frame 50.

### Sixth Embodiment

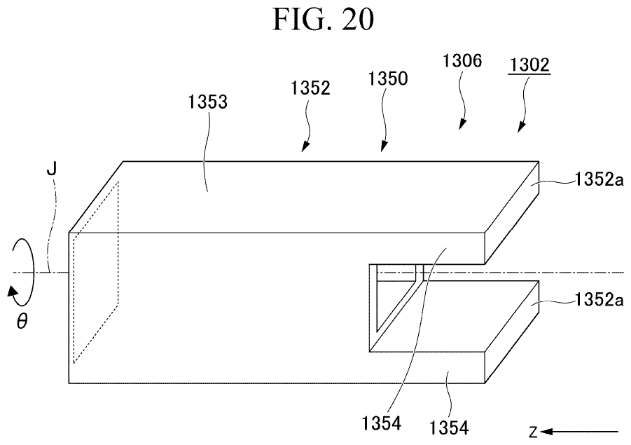

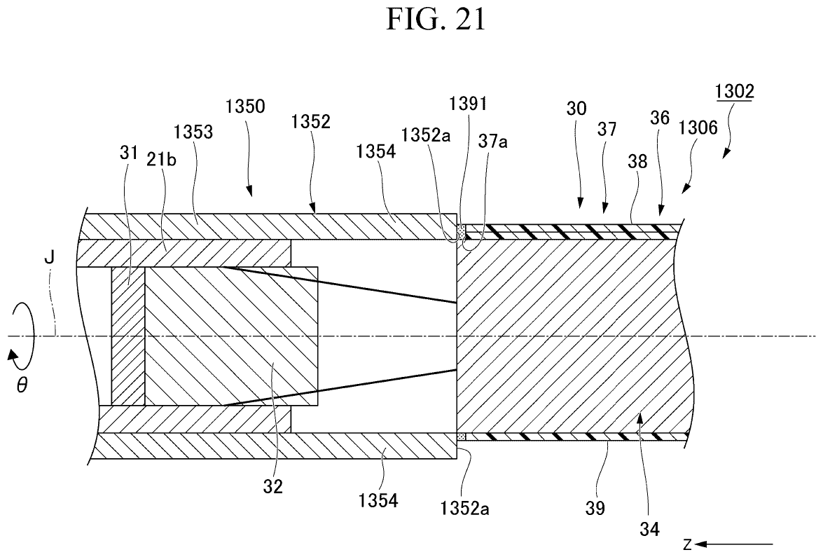

FIG. 20 is a perspective view showing a unit holding frame 1352 of the present embodiment. FIG. 21 is a cross-sectional view showing a part of an imaging unit 30 of the present embodiment. In the present embodiment, an insertion section 1306 of an endoscope 1302 includes a holding frame 1350. In the following description, the same constituent elements in aspect as those in the first embodiment described above are designated by the same reference signs, and the description thereof will be omitted.

The holding frame 1350 of the present embodiment has a lens barrel (not shown) and a unit holding frame 1352. The configuration and the like of the lens barrel of the present embodiment are the same as those of the lens barrel 51 of the first embodiment described above. As described in FIG. 20, the unit holding frame 1352 has a tubular shape centered on the central axis J. The unit holding frame 1352 is open on both sides in the optical axis direction. As described in FIG. 21, the unit holding frame 1352 surrounds the imaging unit 30 from the outside in the radial direction. As described in

FIG. 20, the unit holding frame 1352 has a main body portion 1353, a protrusion 1354, and a facing surface 1352a. That is, the holding frame 1350 has the main body portion 1353 and the protrusion 1354.

The main body portion 1353 is a portion of the unit holding frame 1352 on the object side (+Z side). The main body portion 1353 has a tubular shape, surrounds the central axis J, and extends in the optical axis direction. In the present embodiment, the main body portion 1353 has a substantially tetragonal tubular shape centered on the central axis J and extends in the optical axis direction. Although not shown in the figure, the outer surface of the main body portion 1353 is fixed to the inner surface of the lens barrel 51.

The protrusion 1354 has a plate shape and protrudes from a part of the main body portion 1353 toward the proximal end side (−Z side). In the present embodiment, the unit holding frame 1352 has two protrusions 1354. Each of the protrusions 1354 protrudes toward the proximal end side from different ones of the four wall portions of the main body portion 1353. The plate surface of each protrusion 1354 faces a direction orthogonal to the optical axis direction. The plate surfaces of the protrusions 1354 face the same direction. The protrusions 1354 are spaced apart from each other. The number of protrusions 1354 that the unit holding frame 1352 has may be one or three. In the present embodiment, the unit holding frame 1352 has two facing surfaces 1352a. Each of the facing surfaces 1352a is a portion of the outer surface of one of the different protrusions 1354 which faces the proximal end side.

As described in FIG. 21, a distal end 37a of a protective member 36 faces each of the protrusions 1354 in the optical axis direction. In the present embodiment, the distal end 37a is adhered to each of the facing surfaces 1352a with an adhesive 1391. In other words, the distal end 37a is adhered to each of the protrusions 1354. In this way, the distal end 37a is adhered to the holding frame 1350. In other words, the protective member 36 is adhesively fixed to the holding frame 1350.

According to the present embodiment, the holding frame 1350 has the main body portion 1353 that has a tubular shape and extends in the optical axis direction and the protrusion 1354 that protrudes from a part of the main body portion 1353 toward the proximal end side (−Z side), and the distal end 37a of the protective member 36 faces the protrusion 1354 in the optical axis direction and is adhered to the protrusion 1354. For this reason, in the signal cable 34, the distal end portion 37, the adhesive 1391, the protrusion 1354, and the main body portion 1353, a change in rigidity from the proximal end side toward the object side can be made more gradual. Therefore, even though bending stress is applied to the signal cable 34 when a bending portion 12 is bent, the distal end portion 37, the adhesive 1391, and the protrusion 1354, and the main body portion 1353 are elastically deformed in stages, and thus it is possible to more suitably prevent the bending stress from concentrating on the signal cable 34. Therefore, it is possible to more suitably prevent the signal cable 34 from being damaged.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

The configuration or the like of the protective member is not limited to that of the present embodiment, and for example, the protective member may be made of a metal. Furthermore, the protective member may have another shape such as an angular tube shape and extends in the optical axis direction.

The holding frame does not have to have the lens barrel. In this case, the holding frame is constituted by only the unit holding frame, and the protective member is adhered to the unit holding frame. As a result, the protective member is adhesively fixed to the holding frame.

What is claimed is:

1. An endoscope comprising:
an imaging unit; and
a holding frame having a tubular shape, holding the imaging unit,
a signal cable extending from the imaging unit, and
a protective member having a tubular shape, surrounding the signal cable, and extending in the optical axis direction,
wherein a distal end of the protective member faces the holding frame in the optical axis direction and is adhered to the holding frame,
wherein a distal end portion of the protective member which is a portion on an object side including the distal end has a first distal end portion and a second distal end portion,
wherein the first distal end portion is a part of the distal end portion in a circumferential direction, and the second distal end portion is another part of the distal end portion in the circumferential direction, and
wherein a thickness of the first distal end portion in a radial direction is greater than a thickness of the second distal end portion in the radial direction.

2. The endoscope according to claim 1, further comprises
an imaging board is disposed in the holding frame, and
wherein an end portion of the signal cable on the object side is joined to the imaging board.

3. The endoscope according to claim 2,
wherein the holding frame has an opening portion that is open on a proximal end side, and
wherein the signal cable is inserted into the holding frame through the opening portion.

4. The endoscope according to claim 1,
wherein the protective member is made by forming a sheet member having a sheet shape and extending in the optical axis direction into a tubular shape, and
wherein the first distal end portion is a portion of the distal end portion in which portions of the sheet member overlap each other in the radial direction.

5. The endoscope according to claim 1,
wherein the protective member is formed by folding a folded portion, which is a part of the tubular member having a tubular shape and extending in the optical axis direction in the circumferential direction, in the circumferential direction, and fixing the folded portion and a portion other than the folded portion to each other, and
wherein the first distal end portion is a portion of the distal end portion which includes the folded portion.

6. The endoscope according to claim 1,
wherein the protective member has a first protective member having a tubular shape, surrounding the signal cable, and extending in the optical axis direction, and a second protective member in contact with a part of an outer circumferential surface of the first protective member in the circumferential direction, and
wherein the first distal end portion is constituted by a portion of the first protective member which overlaps the second protective member in the radial direction, and the second protective member.

7. The endoscope according to claim 1,
wherein the protective member has a first protective member having a tubular shape, surrounding the signal cable, and extending in the optical axis direction, and a second protective member in contact with a part of an inner circumferential surface of the first protective member in the circumferential direction, and
wherein the first distal end portion is constituted by a portion of the first protective member which overlaps the second protective member in the radial direction, and the second protective member.

8. The endoscope according to claim 1,
wherein the imaging unit has a binding member that is bound to the protective member, and
wherein the protective member and the signal cable are fixed to each other by the binding member.

9. The endoscope according to claim 1, wherein the protective member is adhesively fixed to the signal cable.

10. The endoscope according to claim 1,
wherein each of the holding frame and the protective member has a tubular shape centered on a central axis, and
wherein, when viewed in the optical axis direction, a difference between a minimum distance between the central axis and an inner surface of the protective member and a minimum distance between the central axis and an inner surface of the holding frame is less than or equal to the thickness of the second distal end portion in the radial direction.

11. The endoscope according to claim 1, wherein the imaging unit has an elastic member that is disposed between the distal end portion and the signal cable.

12. The endoscope according to claim 1, wherein the imaging unit has an elastic member that covers the distal end portion from the outside in the radial direction.

13. The endoscope according to claim 1, further comprising an insertion section that is inserted into a subject,
wherein the imaging unit, the holding frame, and a plurality of accommodation objects are accommodated in the insertion section, and
wherein the first distal end portion faces at least one of the plurality of accommodation objects in the radial direction.

14. The endoscope according to claim 1, further comprising an insertion section that is inserted into a subject,
wherein the insertion section has a case that accommodates the imaging unit and the holding frame, and
wherein the first distal end portion faces the case in the radial direction.

15. The endoscope according to claim 1, wherein, when viewed in the optical axis direction, the protective member has a circular ring shape and the holding frame has an octagonal ring shape.

16. The endoscope according to claim 1, wherein, when viewed in the optical axis direction, each of the protective member and the holding frame has a circular ring shape.

17. The endoscope according to claim 1,
wherein the holding frame includes a unit holding frame that holds the imaging unit and a lens barrel that surrounds the unit holding frame from the outside in the radial direction and holds the unit holding frame, and
wherein the distal end of the protective member faces the lens barrel in the optical axis direction and is adhered to the lens barrel.

18. The endoscope according to claim 1,
wherein the holding frame has a main body portion that has a tubular shape and extends in the optical axis direction and a protrusion that protrudes from a part of the main body portion toward a proximal end side, and
wherein the distal end of the protective member faces the protrusion in the optical axis direction and is adhered to the protrusion.
